(19) United States
(12) Patent Application Publication
(10) Pub. No.: US 2024/0399348 A1
(43) Pub. Date: Dec. 5, 2024

(54) HIGH PERFORMANCE PHOSPHINAMINE COMPLEX SUPPORTED METAL-ORGANIC FRAMEWORK FOR ETHYLENE DIMERIZATION

(71) Applicant: Qatar Foundation for Education, Science and Community Development, Doha (QA)

(72) Inventors: Sherzod MADRAHIMOV, Doha (QA); Elumalai PALANI, Doha (QA); Hind MAMLOUK, Doha (QA); Wenmiao CHEN, Doha (QA)

(21) Appl. No.: 18/724,848

(22) PCT Filed: Dec. 28, 2022
(86) PCT No.: PCT/QA2022/050025

§ 371 Date: Jun. 27, 2024

Related U.S. Application Data
(60) Provisional application No. 63/294,661, filed on Dec. 29, 2021.

(51) Int. Cl. B01J 31/16; B01J 31/18

(52) U.S. Cl. CPC B01J 31/1691; B01J 31/188; B01J 2231/20; B01J 2531/0216; B01J 2531/847

(57) ABSTRACT

A catalyst for ethylene dimerization is provided. The catalyst includes a metal-organic framework; a phosphinamine ligand; and a salt.

### PRIORITY CLAIM

The present application claims priority to and the benefit of U.S. provisional patent application No. 63/294,661, filed on Dec. 29, 2021, the entirety of which is incorporated herein by reference.

### BACKGROUND

Ethylene oligomerization is a widely used process that uses a catalyst to polymerize ethylene to a finite degree. The catalyst used in ethylene oligomerization may effect the reactivity, selectivity, and stability of the reaction. Currently, commercial catalysts cannot produce 1-butene, a product of the reaction, in high purity. Further, commercial catalysts are not effective under gas phase and continuous flow conditions. Thus, improved catalysts for ethylene dimerization are needed.

### SUMMARY

The present disclosure generally relates to a catalyst for ethylene dimerization.

In light of the present disclosure, and without limiting the scope of the disclosure in any way, in an aspect of the present disclosure, which may be combined with any other aspect listed herein unless specified otherwise, a catalyst for ethylene dimerization is provided. The catalyst includes: (1) a metal-organic framework (e.g., UiO-66); (2) a phosphinamine ligand (e.g., a Bis(diaryl/dialkylphosphino) amine ligand); and (3) a salt (e.g., a Ni salt).

The reader will appreciate the foregoing details, as well as others, upon considering the following detailed description of certain non-limiting embodiments according to the present disclosure.

### DETAILED DESCRIPTION

The present disclosure generally relates to a catalyst for ethylene dimerization.

Currently, commercial catalysts cannot produce 1-butene, a product of the reaction, in high purity. Further, commercial catalysts are not effective under gas phase and continuous flow conditions. Thus, aspects of the present disclosure may address the above-discussed constraints in conventional catalysts for ethylene dimerization.

According to an embodiment of the present disclosure, the catalyst may include three different components: (1) a metal-organic framework (e.g., UiO-66); (2) a phoshphinamine ligand (e.g., a Bis(diaryl/dialkylphosphino amine ligand); and (3) a salt (e.g., Ni salt).

In the present embodiment, the metal-organic framework (e.g., UiO-66) is synthesized through hydrothermal methods using, for example, terephthalic acid and Zirconyl chloride. Further, the Bis(diaryl/dialkylphosphino) amine ligand and Ni metalation is synthesized through solvent assist ligand exchange and metalation according to an embodiment.

The present embodiment maintained high activity and exclusive selectivity for 1-butene and can produce 1-butene gas in high purity and work under gas-phase and continuous flow conditions.

According to an embodiment, MOF-immobilized molecular Ni Bis(diaryl/dialkylphosphino) amine complexes for ethylene oligomerization reactions can be performed under mild conditions. The amine complexes include metal-organic framework (e.g., Ui0-66); a phosphinamine ligand (e.g., a Bis(diaryl/dialkylphosphino amine ligand); and a salt (e.g., Ni salt). To synthesize the catalyst, several additional materials are required, such as terephthalic acid; Zirconyl chloride; 2-Aminoterephthalic acid; Chlorodiiso propylphosphine; and Nickel(ll) chloride ethylene glycol dimethyl ether complex. In an embodiment, the immobilized catalyst is prepared through a post-synthetic functionalization (PSF) of metal-organic framework (MOF) zirconium (IV) based UiO66 MOF, via a ligand exchange method followed by metalation by Ni. A series of PNP-R1 (R=-iPr, -tBu, -Cy, -Ph and -Et) ligands with varying electronic and steric properties were screened to determine the most active catalyst for the ethylene dimerization reaction. Unlike the current homogeneous catalytic process in solution, the catalyst can produce 1-butene gas in high purity, and work under gas-phase and continuous flow conditions.

According to an embodiment, the metal-organic framework (MOF) is synthesized through hydrothermal methods with terephthalic acid and Zirconyl chloride. The Bis(diaryl/dialkylphosphino) amine ligand and Ni metalation is synthesized through solvent assist ligand exchange and metalation. The present disclosure is based on the Bis(diaryl/dialkylphosphino) amine ligand, for example, that has the potential for ethylene oligmerization in industrial application. According to an embodiment, the heterogeneous catalyst possesses high reactivity (e.g., TOF higher than 40000 (mol ethylene)/(mol Ni·h)), selectivity (e.g., over 99% 1-butene product) and high stability, where all parameters are comparable with the best reported catalysts.

Compared with porous silica, MOFs provide a number of advantages. For example, MOFs are highly ordered porous crystalline materials with extraordinary tenability of the structure. When molecular catalysts are immobilized on MOFs, due to the crystalline nature of the MOF supports, the exact location and the content of the catalyst loading is known, which is hard to predict for amorphous catalysts, such as silica. Further, active sites are uniformly distributed throughout the support surface. The size of MOFs particles and porosity can be tuned from around 20 nm to mm scale, which allows operability of the dimerization in a microreactor under flow conditions and in gas phase.

Using a phosphinamine complex in the catalyst provides a number of advantages. For example, the strong donating phosphine group is more active (e.g., 29000 TOF at 15 bar) compared to, for example, Ni-MFU-41 (11100 TOF at 15 bar). It is easy to synthetically modify the phosphine ligands used in the catalyst, thus providing the ability to tune the electronic and steric properties of the catalyst thereby resulting in optimum activity and selectivity. Further, the high tunability of UiO66, for example, enables synthesizing MOF particles with a designated size for packing in flow microreactor and run the reaction under flow condition and in gas phase

Immobilizing the molecular catalysts on the MOF provides a number of advantages over a homogeneous system according to an embodiment. For example, heterogenization of the catalyst allows for the separation of the catalyst from the product with ease and recycle of the catalyst. Heterogenization also stabilizes the catalyst and allows for the reaction run under gas phase. This is not possible for a homogeneous catalyst. Heterogenization also allows for utilization of the catalyst in the microreactor continuous production in gas and liquid phase.

As a result, the catalyst (e.g., a UiO66-PNP-Ni catalyst) showed systemic varying of reactivity when manipulating the electronic and steric properties. In a preferred embodiment, a UiO66-PNPiPr-Ni catalyst provided an optimized reactivity of 29000 TOF at 15 bars, which is comparable with homogeneous phosphine Ni complexes and higher than most of solid catalysts. Moreover, the catalyst reaction can be performed under pure gas phase and in the microreactor under flow conditions for continuous reaction.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. A catalyst for ethylene dimerization comprising:
a metal-organic framework;
a phosphinamine ligand; and
a salt.

2. The catalyst according to claim 1, wherein the metal-organic framework is UiO66.

3. The catalyst according to claim 1, wherein the phosphinamine ligand is a Bis(diaryl/dialkylphosphino) amine ligand.

4. The catalyst according to claim 1, wherein the phosphinamine ligand is a PNP-R2 ligand, where R is -iPr, -tBu, -Cy, -Ph or -Et.

5. The catalyst according to claim 4, wherein the catalyst is a UiO66-PNPiPr-Ni catalyst.

6. The catalyst according to claim 1, wherein the salt includes a Ni salt.

7. The catalyst according to claim 1, wherein the catalyst has a reactivity at a TOF higher than 40000 (mol ethylene)/(mol Ni·h).

8. The catalyst according to claim 1, wherein the catalyst has a selectivity greater than 99% 1-butene product.
